(19) United States
(12) Patent Application Publication
(10) Pub. No.: US 2023/0270084 A1
(43) Pub. Date: Aug. 31, 2023

(54) SYSTEM AND METHOD FOR USING ANIMAL DNA TO PREDICT OFFSPRING PHENOTYPE AND TO OPTIMIZE DIETARY REQUIREMENTS

(71) Applicants: Devin George Fausett, Hooper, UT (US); Krista Fisher Fausett, Hooper, UT (US)

(72) Inventors: Devin George Fausett, Hooper, UT (US); Krista Fisher Fausett, Hooper, UT (US)

(21) Appl. No.: 18/176,408

(22) Filed: Feb. 28, 2023

Related U.S. Application Data
(60) Provisional application No. 63/314,997, filed on Feb. 28, 2022.

(51) Int. Cl. A01K 67/02; G16B 20/00; G06T 11/00; G06F 16/532

(52) U.S. Cl. CPC A01K 67/02; G06F 16/532; G06T 11/00; G16B 20/00; A01K 2227/10

(57) ABSTRACT

The present invention is a system and method for using DNA markers and advances in genetic phenotyping information to obtain canine offspring having a desired phenotype, wherein the genetic information of a canine sire or dam, the known parent, is used in combination with the desired phenotype of the offspring to thereby determine the necessary DNA markers of the unknown canine parent, generating a visual illustration of the unknown canine parent, and then searching through a database of potential sires and dams to identify a dam or sire that most closely resembles the predicted unknown canine parent.

### BACKGROUND

Field of the Invention: This invention relates generally to the selected breeding and care of animals. More specifically, the invention is directed to the breeding of dogs by using a database of canine DNA or genotypes to determine the physical traits or phenotype of offspring that would be the result of the breeding of a particular canine sire and dam, where the breeder knows the desired phenotype of the offspring and has the DNA of either a sire or a dam and uses the invention to determine the closest match of the other parent in a database of canine DNA. Once the offspring is produced, the dietary requirements are then optimized based on the genetics of the sire and dam.

Description of Related Art: The prior art includes various examples of how DNA can be used to determine various physical traits such as health risks as the result of breeding of mammals. For example, some prior art teaches how the use of genomic data of a mammal can be used to determine the probability or severity of different traits of offspring, such as disease, morphology and/or behavior traits for determining a value of health or disease risks and/or breeding value of the mammals.

The prior art also teaches using canine DNA to predict the overall appearance, or phenotype, of a canine from its DNA. The DNA phenotyping can be used in forensics to predict a deceased dog’s coat color, coat pattern, coat structure, body size, ear shape, and tail length using known DNA markers. This is useful when trying to determine what a dog looked like that was found at a forensic scene.

A final example of using canine DNA is to determine the likely parentage of a known dog. For instance, the sire and dam of a dog is used to determine the likely breed of the sire and dam within a particular percentage of accuracy.

However, none of the prior art appears to teach how to use known DNA markers as a predictive source of canine phenotyping. Accordingly, it would be an advantage over the prior art to be able to predict the missing phenotype of one of the canine parents when two items are known quantities. These two known quantities are first, the DNA markers of one canine parent, and two, the DNA markers of the desired offspring. In other words, the prior art fails to teach the aspect of how to use DNA markers to establish the physical characteristics of the other canine parent when you begin with a known canine parent, and the user knows what the desired canine offspring should look like.

### BRIEF SUMMARY

The present invention is a system and method for using DNA markers and advances in genetic phenotyping information to obtain canine offspring having a desired phenotype, wherein the genetic information of either a canine sire or dam, the known parent, is used in combination with the desired phenotype of the offspring to thereby determine the necessary DNA markers of the unknown canine parent, generating a visual illustration of the unknown canine parent, and then searching through a database of potential sires and dams to identify a dam or sire that most closely resembles the predicted unknown canine parent. Furthermore, once the canine offspring is born, a diet may be optimized that is based on the region that the dam and sire each originated.

These and other embodiments of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is an example of a Punnett Square.

FIG. 2 is an example of an E Locus chart.

FIG. 3 is an example of a graphic chart showing breeding possibilities using E Locus, K Locus, B Locus, A Locus, D Locus and S Locus.

FIG. 4 is a flowchart of the prior art process of determining phenotype of resulting canine offspring based on genotypes of parents.

FIG. 5 is a flowchart showing the steps of the first embodiment.

FIG. 6 is a flowchart showing the additional steps of generating an image and finding the second canine parent.

FIG. 7 is a flowchart of the second embodiment of the invention.

FIG. 8 is a flowchart showing the optimization of offspring diet through selecting foods from region that breed of offspring originated.

### DETAILED DESCRIPTION

Reference will now be made to the drawings in which the various embodiments of the present invention will be discussed so as to enable one skilled in the art to make and use the invention. It is to be understood that the following description illustrates embodiments of the present invention and should not be viewed as narrowing the claims which follow.

This document will refer to phenotypes of offspring and parents of mammals. As is known to those skilled in the art, a phenotype is an animal’s observable traits, such as height, eye color, color of coat, etc. as well as development, and behavior. The combination of alleles a dog has is known as the genotype. The physical characteristics a dog has in known as its phenotype. How the genotype (the dog’s genes) influences the phenotype (the way it looks) is not always straightforward. The genetic contribution to the phenotype will be referred to as the genotype. It is known that some traits are largely determined by the genotype, while other traits are largely determined by environmental factors.

Of particular relevance to the embodiments of the invention are those physical, behavioral and health characteristics that might be determined using the current state of the art in animal genetics. Relevant characteristics are not only those that can be determined ahead of birth but also after birth, as will be explained.

The canine breeding business is a big and expensive business. Because of the expense involved, breeders like to be as certain as possible of the outcome of any breeding that occurs with selected sires and dams before breeding takes place. While every breeding outcome can’t be perfectly predicted, it should be no surprise that advancements in genetic technology have removed much of the guesswork from the breeding process.

However, a typical use of genetic information is simply to determine the likely phenotype of offspring of a known sire and dam. Thus, the owners of a particularly desirable sire and dam may want to breed them just for the possibility of obtaining offspring with some combination of the characteristics of the canine parents. Thus, an analysis may be performed to determine the likely phenotype of any offspring given the known genetic information of the sire and the dam.

In contrast, the first embodiment of the present invention approaches the inherent difficulties of dog breeding in a different but extremely desirable manner. Instead of beginning with a known sire and dam and determining the likely phenotype of resulting offspring when breeding them, the first embodiment begins with a single known canine parent. The owner of the known canine parent is not relevant. All that is important is that the canine parent is available for breeding purposes.

The next step is for the breeder to use the first embodiment to identify the phenotype of the desired offspring. In other words, the first embodiment of the invention is used to identify all of the physical characteristics of the desired offspring. It should be understood that the desired offspring must be a possible offspring of the known canine parent. Accordingly, the first embodiment uses as input the genotype of the known canine parent. The first embodiment is then used to identify the phenotype of the desired offspring.

The first embodiment will inform the breeder if a particular selected phenotype of the desired offspring is not possible. This may be accomplished, for example, by not allowing a particular phenotype of the desired offspring to be a selectable option.

One method to accomplish this in the first embodiment may be through the use of a genetic analysis computer program. The genetic analysis computer program may include drop down menus that list all possible phenotype options. These phenotype options may then be selectively made available to the user of the genetic analysis computer program based on the known canine parent.

For example, a breeder may desire the offspring to have a particular color of coat. The genetic analysis computer program receives as input the genotype of the known canine parent. Based on the one known canine parent, not all color coats may be possible for the offspring. Thus, the genetic analysis computer program would allow the breeder to see all possible coat colors for the desired offspring given the genotype of the known canine parent. The genetic analysis computer program would hide or otherwise make unavailable all those coat colors that are not possible to obtain given the genotype of the known canine parent.

The breeder would then select all possible phenotypes of the desired offspring given the known limitations imposed by the genotype of the known canine parent. It may be the case that for some phenotype categories, all options may be available, less than all options are available, or only one option is available.

Once the desired phenotype of the desired offspring has been entered into the first embodiment of the genetic analysis computer program, the next step is to perform a unique feature of the first embodiment which is the identification of the unknown parent canine. The last step may be accomplished by using the first embodiment to combine the genotype of the known canine parent and the phenotype that has just been selected for the desired canine offspring.

The first embodiment may perform this task when the genetic analysis computer program uses the data that was already input for the known canine parent and the data that the breeder has input for the phenotype of the desired canine offspring. The output of this step may be an illustration of the unknown canine parent.

The illustration of the unknown canine parent generated by the first embodiment of the invention may be generated in any convenient manner. For example, the illustration may appear as a computer generated image. The computer generated image may be a composite image that is compiled from a database of illustrations of all the possible phenotypes of a canine.

In an alternative embodiment, the database may be comprised of a plurality of pictures of canines, wherein each of the canines is categorized by phenotypes. After the genetic analysis computer program has determined the phenotypes of the unknown parent, the genetic analysis computer program may search through the database to locate a picture of an actual canine that has all or a particular number of the phenotypes. The genetic analysis computer program may then identify for the breeder the phenotypes that are not an exact match for the desired phenotypes in the picture from the database.

What is important is that an image, either an actual picture or a composite that is constructed from all of the desired phenotypes, is generated by the first embodiment of the invention. Generating the image is important because it enables the breeder to have an illustration of the best candidate for the unknown canine parent. This candidate for the unknown canine parent must then be located by the breeder.

In the first embodiment of the invention, the breeder may then use the generated image to locate a possible breeding candidate as the unknown canine parent.

The first embodiment may offer another a breeding database of pictures of available canine breeding candidates. This breeding database may be compiled by people who are offering their canines as breeding partners.

Alternatively, the breeder may post the generated image on a dog breeding forum asking for anyone with a canine that looks like the image to contact the breeder.

Thus, the first embodiment of the invention may provide a new way to use the newly identified phenotype of the unknown canine parent in the form of the generated image to assist the breeder in locating a suitable breeding match to obtain the desired canine offspring.

This may be accomplished in the first embodiment by establishing a database of genetic information for sires and dams. Using the genetics of a known dam, the first embodiment of the invention may use an algorithm to match the known dam to one or more sires in the genetic database to create the desired phenotype outcome in offspring. The desired offspring should not only have the phenotype of the parent canines but should also have desirable qualities of health in order to achieve responsible, ethical, and intentional breeding practices.

Unfortunately, it is often the case that a dog breeder may find that it is necessary to compromise and select physical traits over health, or health over physical traits. However, by providing a genetic database of sires and dams, the first embodiment may be used to determine the best parentage to obtain a desired phenotype in offspring while also obtaining healthy offspring.

The genetic database may include genetic profiles and health profiles for each sire and dam that may serve as the unknown parent canine. A breeder of a particular sire or dam may then input the desired phenotype outcome for offspring. In the first embodiment, the genetic analysis computer program then matches the known dam or sire with an unknown dam or sire in the genetic database that will result in the desired offspring.

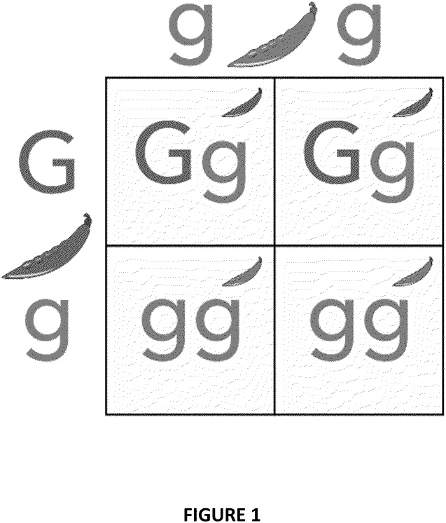

All genetic possibilities are calculated using Punnett squares, as shown in FIG. 1.

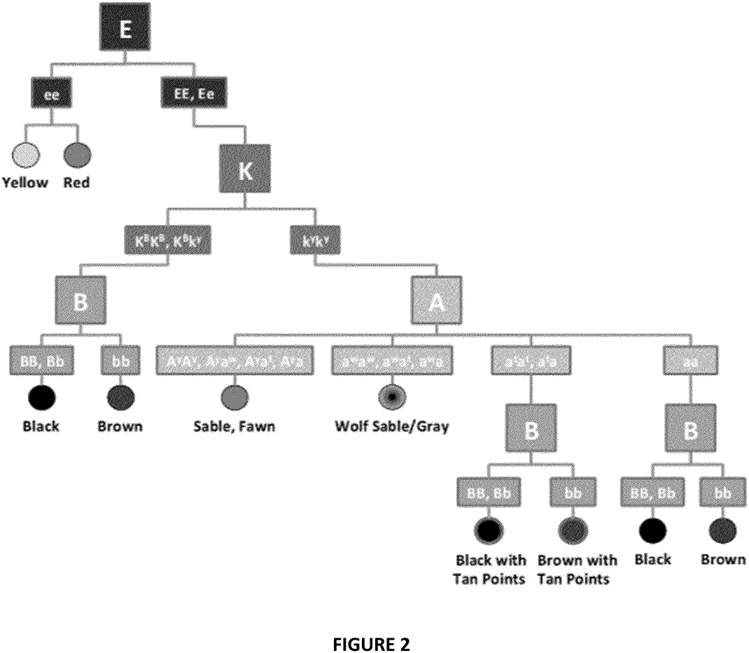

FIG. 2 is provided as an E Locus chart showing possibilities of breeding outcomes.

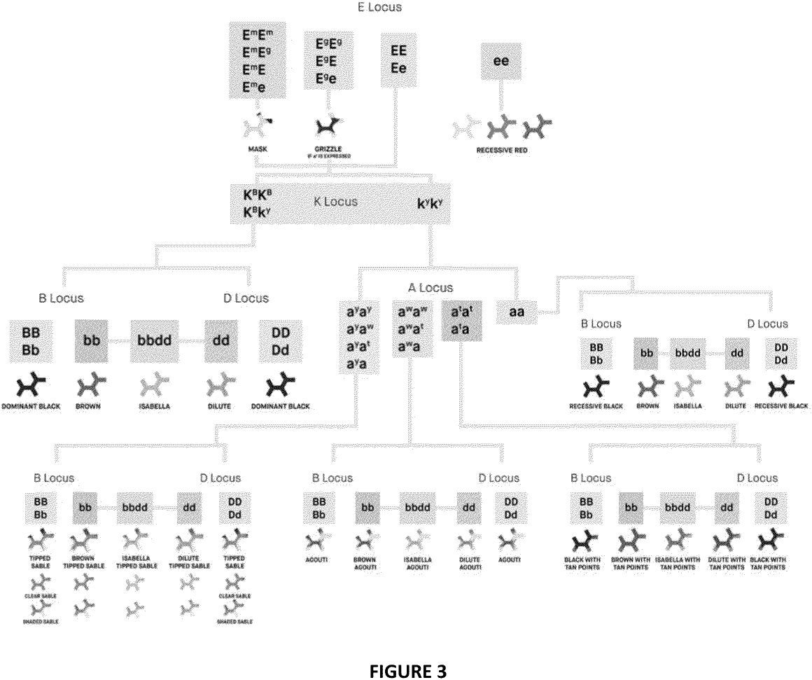

FIG. 3 is a graphic chart showing breeding possibilities using E Locus, K Locus, B Locus, A Locus, D Locus and S Locus.

When determining coat color, always begin with the E locus.
Em - (Mask) Dominant over Eg, E and e
Eg - (Grizzled) Dominant over E and e
E - Dominant over e
e - (Recessive Red) Recessive

Table 1 is a chart showing possible E Locus Genetic outcomes of Breeding.

TABLE 1

EmEm × EmEm = 100% EmEm
EmEm × EmEg = 50% EmEm / 50% EmEg
EmEm × EmE = 50% EmEm / 50% EmE
EmEm × Eme = 50% EmEm / 50% Eme
EmEm × EgEg = 100% EmEg
EmEm × EgE = 50% EmEg / 50% EmE
EmEm × Ege = 50% EmEg / 50% Eme
EmEm × EE = 100% EmE
EmEm × Ee =50% EmE / 50% Eme
EmEm × ee = 100% Eme
EmEg × EmEg = 25% EmEm / 50% EmEg / 25% EgEg
EmEg × EmE = 25% EmEm / 25% EmEg / 25% EmE / 25% EgE
EmEg × Eme = 25% EmEm / 25% Eme / 25%EmEg / 25% Ege
EmEg × EgEg = 50% EmEg / 50% EgEg
EmEg × EgE = 25% EmEg / 25% EgEg / 25% EmE / 25% EgE
EmEg × Ege = 25% EmEg / 25% EgEg / 25% Eme / 25% Ege
EmEg × EE = 50% EmE / 50% EgE
EmEg × Ee = 25% EmE / 25% EgE / 25% Eme / 25% Ege
EmEg × ee = 50% Eme / 50% Ege
EmE × EmE = 25% EmEm / 25% EmE / 25% EE
EmE × Eme = 25% EmEm / 25% EmE / 25% Eme / 25% Ee
EmE × EgEg = 50% EmEg / 50% EgE
EmE × Ege = 25% EmEg / 25% EgE / 25% Eme / 25% Ee
EmE × EgE = 25% EmEg / 25% EgE / 25% EmE / 25% EE
EmE × EE = 50% EmE / 50% EE
EmE × Ee = 25% EmE / 25% EE / 25% Eme / 25% Ee
EmE × ee =50% Eme / 50% Ee
Eme × Eme = 25% EmEm / 50% Eme / 25% ee
Eme × EgEg = 50% EmEg / 50% Ege
Eme × EgE = 25% EmEg / 25% Ege / 25% EmE / 25% Ee
Eme × Ege = 25% EmEg / 25% Ege / 25% Eme / 25% ee
Eme × EE = 50% EmE / 50% Ee
Eme × Ee = 25% EmE / 25% Ee / 25% ee /Eme
Eme × ee = 50% Eme / 50% ee
EgEg × EgEg = 100% EgEg
EgEg × EgE = 50% EgEg / 50% EgE
EgEg × Ege = 50% EgEg / 50% Ege
EgEg × EE = 100% EgE
EgEg × Ee =50% EgE / 50% Ege
EgEg × ee = 100% Ege
EgE × EgE = 25% EgEg / 50% EgE / 25% EE
EgE × Ege = 25% EgEg / 25% EgE / 25% Ege / 25% Ee
EgE × EE = 50% EgE / 50% EE
EgE × Ee = 25% EgE / 25% EE / 25% Ege / 25% Ee
EgE × ee = 50% Ege / 50% Ee
Ege × Ege = 25% EgEg / 50% EgE / 25% ee
Ege × EE = 50% Ege / 50% Ee
Ege × Ee = 25% EgE / 25% Ee / 25% Ege / 25% ee
Ege × ee = 50% Ege / 50% ee
EE × EE = 100% EE
EE × Ee = 50% EE / 50% Ee
EE × ee = 100% Ee
Ee × Ee = 25% EE / 50% Ee / 25% ee
Ee × ee = 50% Ee / 50% ee
ee × ee = 100 % ee

The following observations are noted for Table 1. For the following breeding pairs with the listed outcome ee × ee = 100% will be shades from yellow to red. Ee × Ee = 25% ee will be shades from yellow to red (See intensity locus for shade of red. (This is the only time the intensity locus applies).

Then See S locus for possible addition of white. See B locus for Nose and paw pads coloring. ee + intensity locus + S locus + B locus will determine final coat color. For the following breeding pairs, see K locus

$$\begin{matrix}\text{EE} \times \text{EE} = 100 \% \text{EE} \\ \text{EE} \times \text{ee} = 100 \% \text{Ee} \\ \text{Ee} \times \text{EE} = 50 \% \text{Ee} / 50 \% \text{EE} \\ \text{ee} \times \text{Ee} = 50 \% \text{Ee} \\ \text{Ee} \times \text{Ee} = 25 \% \text{EE} / 50 \% \text{ee}\end{matrix}$$

The problem facing breeders is finding a canine parent for another canine parent to produce the desired offspring with both the traits and health needed for responsible, ethical and intentional breeding practices. Breeders may search for hours through listing sites, contacting other breeders, going over genetics and matching dams to the sires each time a particular dam is in heat. It is time consuming, limited, frustrating and unpredictable. Often, the decision facing breeders is to compromise traits for health or health for traits so that the breeders can continue to run a business.

The first embodiment of the invention may be a website. The first embodiment utilizes a genetic analysis computer program to match breeding canine parents (dams and sires) based on which combination will produce the desired offspring. This will be accomplished by creating genetic and health profiles for the breeding dogs. The breeder will then input the desired outcome for the canine offspring and the genetic analysis computer program will match the dam to the sire that is the best match followed by alternatives ranked from best match down least desired match.

For example, a breeder has a Cavalier King Charles Spaniel Dam that weighs 14 lbs., has a Blenheim coat and carries 2 copies, but is not affected by IVDD. The breeder would like this dam to have a litter of F1 Cavapoo puppies with wavy, fluffy coats who are not affected by IVDD and average 12 lbs. as adults.

The breeder has selected the use of Transcervical Insemination and based on the results of progesterone testing, will need to be bred in the next 24-48 hours. The breeder would then like the litter to have the possibility of having a puppy with a Blenheim coat, another with a ruby coat, another with a tricolor coat and another with a black with tan point coat.

It is known that the Blenheim dam is ee, atat, kyky, spsp, ff, tt. It is also known that she carries two copies of IVDD and is 14 lbs. Using the algorithm of the first embodiment, based on the genetics and the information we have for the dam and the desired outcome of the litter, it can be determined using the genetic analysis computer program of the first embodiment that the sire would need to be: Ee, atat, kyky, ssp, FF, CC. To safely breed the cavalier without having affected puppies, the poodle would need to be clear for IVDD and weigh 8.5 lbs. or less and must be able to collect and ship semen overnight.

The algorithm would generate a list of all of the sires listed in the database that is accessible through the website who matches that request to give the desired outcome in ranked order from best match down.

Additionally the first embodiment will provide education, calendars, scheduling, forms and databases for breeders to input data that will generate reports.

Sires Specific. Using Sire genetic reports and health testing certifications, a profile may be created for the sire that will be used to match it to a dam based on the desired traits of the canine offspring. The website may include listings for sire services with a profile that can be shared to the dam, a scheduling calendar with alerts and reminders, the ability to create “dummy dams” and “dummy sires” for future litter planning and program growth, program planning and litter planning forms, Best Practices, care and additional education, a reproductive Veterinarian database, and ratings and feedback on the matched dam breeder.

Dam Specific. Using their genetic reports and health testing certifications, we will create a profile for the dam that will be matched to sires in the database that will produce the desired traits in the puppies, a scheduling calendar with alerts and reminders, a Due Date Calculator, and breeding specific reminders and database that will collect data and generate dam specific reports over multiple breedings.

For example, progesterone testing results with a graph that shows cycles overlapping from multiple heats. It will also include the ability to create “dummy dams” and “dummy sires” for future litter planning and program growth, program planning and litter planning forms, Best Practices, care and additional education, a reproductive Veterinarian database, and ratings and feedback on the matched sire breeder.

Pup Specific. Forms and data collection that will generate reports, a scheduling calendar with alerts and reminders, and an educational manual on running a breeding program from the day you bring a dog into your program until they are retired.

The website may also include breeder information and education on everything a breeder needs to run a successful breeding program.

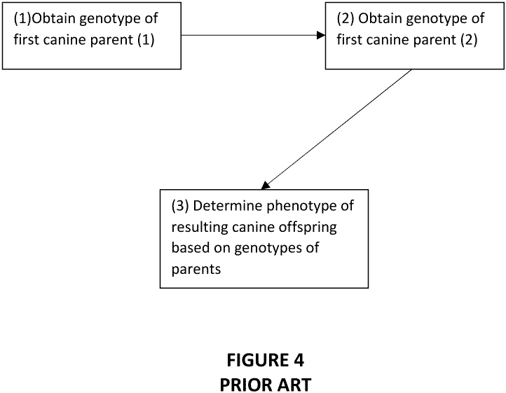

It should be understood that the first embodiment of the invention is not suggesting that the ability to determine the phenotype outcome of breeding a dam and a sire is new. This is known to those skilled in the prior art, is shown in FIG. 4, and does not need to be taught herein. What may be a novel aspect of the first embodiment is that the first embodiment operates in a backwards direction for dog breeding.

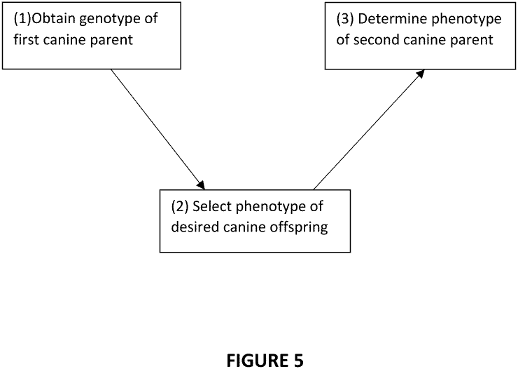

As shown in FIG. 5, instead of taking the genotype of two known canine parents, a dam and a sire, and then determining the likely phenotype of resulting canine offspring, the first embodiment begins with one known parent, either a dam or a sire. The genotype of this known parent is entered into the genetic analysis computer program of the first embodiment.

Next in FIG. 5, the breeder identifies the desired phenotype of the canine offspring by selecting the desired phenotype in the genetic analysis computer program.

Next in FIG. 5, the genetic analysis computer program identifies the phenotype of the unknown parent instead of identifying the phenotype of the canine offspring. It is this aspect of the invention that may be unknown in the prior art.

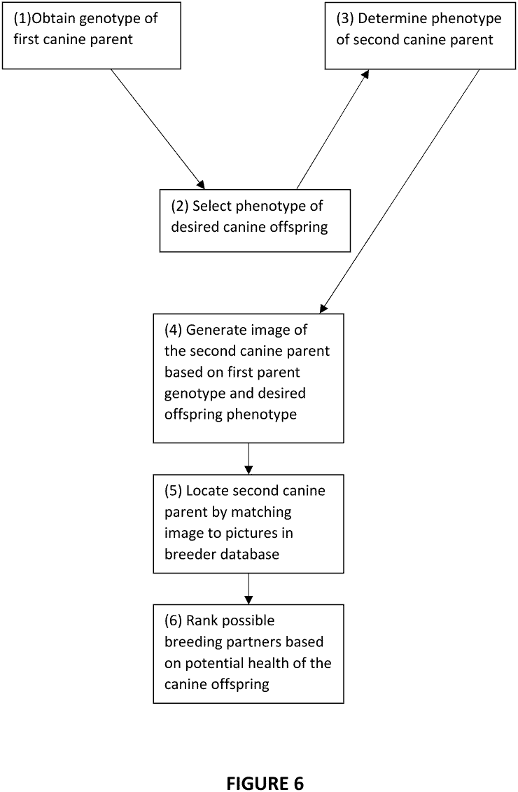

FIG. 6 shows that once the genetic analysis computer program has identified the phenotype of the unknown canine parent, the first embodiment of the invention may generate the image of the unknown canine parent from the identified phenotype. As explained previously, the image may be a composite image of the various portions of the phenotype. For example, the phenotype of the unknown parent has a coat with color A, of length B, etc.

FIG. 6 shows that the breeder may then use a breeder database with images of possible breeding partners to identify the breeding partner that most closely resembles the phenotype image.

If there is more than one possible breeding partner based on the phenotype image of the unknown canine parent, then the first embodiment of the present invention may rank the possible breeding partners based on the potential health of the canine offspring using the genotype data of the possible breeding partners. It is known that genetic testing may reveal potential health issues of any resulting offspring. Accordingly, these factors may be used to highlight any potential health issues that may be revealed through genetics.

The genetic analysis computer program of the first embodiment of the invention may be an algorithm that is accessible through the breeding website. The website may enable a breeder to upload genotype information for their known canine parent. The website may enable the breeder to select the phenotype of the desired canine offspring. The website may also generate an image of the unknown canine parent. Finally, the website may enable a breeder to obtain a ranking of possible breeding partners based on the health of the desired canine offspring based on the genotype of the possible breeding partners when combined with the genotype of the known canine parent.

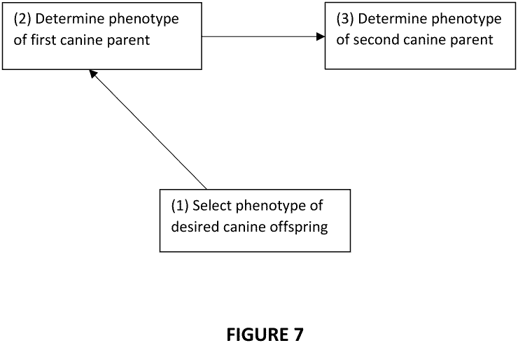

In a second embodiment of the invention shown in FIG. 7, a breeder may begin the process of identifying canine parents by first identifying the phenotype of the desired canine offspring, and then working backwards to identify both canine parents.

Thus, the breeder would use the phenotype of the desired canine offspring to select a first canine parent that may generate the desired canine offspring. The genetic analysis computer program may then generate an image of the first canine parent. This image may then be compared to the breeder database to select the first canine parent.

Having selected the first canine parent, the choices for the second canine parent are now more limited. In the second embodiment of the invention, the genetic analysis computer program may now generate an image of the second canine parent. Again, this image may then be compared to the breeder database to select the second canine parent.

So far, the first and second embodiments teach steps that are followed that lead to the selection of the dam and the sire for a desired canine offspring. The first and second embodiments are also directed to care of the canine offspring after it is born.

There is increasing evidence of a correlation between a canine diet and the origin of the breed or breeds that created the canine offspring. The evidence suggests that the best diet for a canine may be one that is selected from the region or regions from which the breed or breeds come from that are the genotype of the canine. A region may be any geographical area that corresponds to any area that defines where a breed of canine is supposed to have originated.

Prior to being domesticated, dogs, being canines, fended for themselves and survived on a carnivorous diet. After adapting them for protection, work, and companionship, people began to care at least in part for their nutritional needs. Dog food is food specifically formulated and intended for consumption by dogs and other related canines. Dogs are considered to be omnivores with a carnivorous bias. They have the sharp, pointed teeth and shorter gastrointestinal tracts of carnivores, better suited for the consumption of meat than of vegetable substances, yet also have ten genes that are responsible for starch and glucose digestion, as well as the ability to produce amylase, an enzyme that functions to break down carbohydrates into simple sugars, which is something that obligate carnivores like cats lack. Thus, dogs evolved the ability of living alongside humans in agricultural societies, as they managed on scrap leftovers from humans.

Dogs have managed to adapt over thousands of years to survive on the meat and non-meat scraps and leftovers of human existence and thrive on a variety of foods, with studies suggesting dogs’ ability to digest carbohydrates easily may be a key difference between dogs and wolves.

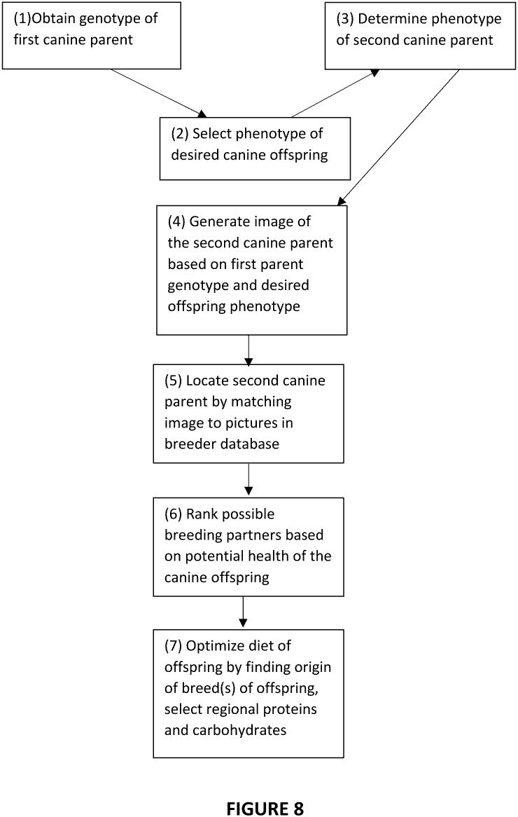

Accordingly, it is another aspect of the embodiments of the invention that when planning a diet for the optimum health of the canine offspring, that the breeder selects dog food that contains proteins and carbohydrates that are from the region where the breed of the canine is a native as shown in FIG. 8.

In summary, the first embodiment of the invention A method for breeding a desired canine offspring with a selected phenotype, said method comprising: obtaining a genotype of a first canine parent, selecting a phenotype of the desired canine offspring, using a genetic analysis computer program to determine a phenotype of a second canine parent based on the phenotype of the desired canine offspring and the genotype of the first canine parent, locating the second canine parent that most closely matches the phenotype of the second canine parent as determined by the genetic analysis computer program, breeding the first canine parent and the second canine parent to obtain the desired canine offspring, and optimizing a diet of the desired canine offspring by selecting food from a region where at least one breed of the first canine parent or the second canine parent originated.

In summary, a system that can perform the method described above is a system for breeding a desired canine offspring with a selected phenotype, said system comprising, a website on the Internet that gives access to a genetic analysis computer program; a database that is accessed by the genetic analysis computer program, wherein the database stores a genetic record or genotype for a plurality of canines that are available for breeding, and wherein the genetic analysis computer program allows a breeder to create a phenotype for a desired canine offspring, wherein the genetic analysis computer program combines a genotype of first canine parent to be used for breeding, and the created phenotype of the desired canine offspring and generates a phenotype of a second canine parent that when bred with the first canine parent will result in the desired canine offspring.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A method for breeding a desired canine offspring with a selected phenotype, said method comprising:
obtaining a genotype of a first canine parent;
selecting a phenotype of the desired canine offspring;
using a genetic analysis computer program to determine a phenotype of a second canine parent based on the phenotype of the desired canine offspring and the genotype of the first canine parent;
locating the second canine parent that most closely matches the phenotype of the second canine parent as determined by the genetic analysis computer program;
breeding the first canine parent and the second canine parent to obtain the desired canine offspring; and
optimizing a diet of the desired canine offspring by selecting food from a region where at least one breed of the first canine parent or the second canine parent originated.

2. The method as defined in claim 1 wherein the method further comprises:
generating an image of the second canine parent based on the first parent genotype and the desired canine offspring phenotype; and
comparing the generated image of the second canine parent to a database of images of potential canine breeding parents.

3. The method as defined in claim 2 wherein the method further comprises creating a database of potential canine parents that contains at least genetic information and a photo of each of the potential canine parents that are stored therein.

4. The method as defined in claim 2 wherein the method of generating the image of the second canine parent further comprises using the genetic analysis computer program to generate a composite image of all of the characteristics that define the phenotype of the second canine parent.

5. The method as defined in claim 4 wherein the method of generating the image of the second canine parent further comprises using the genetic analysis computer program to generate a picture showing a canine having all or most of the characteristics that define the phenotype of the second canine parent.

6. The method as defined in claim 5 wherein the step of locating the second canine parent that most closely matches the phenotype of the second canine parent as determined by the genetic analysis computer program further comprises:
locating a plurality of second canine parent candidates in the database; and
ranking the plurality of second canine parent candidates according to potential health of the canine offspring based on the genetics of the plurality of second canine parent candidates.

7. A system for breeding a desired canine offspring with a selected phenotype, said system comprising:
a website on the Internet that gives access to a genetic analysis computer program;
a database that is accessed by the genetic analysis computer program, wherein the database stores a genetic record or genotype for a plurality of canines that are available for breeding; and
wherein the genetic analysis computer program allows a breeder to create a phenotype for a desired canine offspring, wherein the genetic analysis computer program combines a genotype of first canine parent to be used for breeding, and the created phenotype of the desired canine offspring and generates a phenotype of a second canine parent that when bred with the first canine parent will result in the desired canine offspring.
